(19) United States
(12) Patent Application Publication
(10) Pub. No.: US 2004/0197832 A1
(43) Pub. Date: Oct. 7, 2004

(54) Non-invasive prenatal genetic diagnosis using transcervical cells

(75) Inventors: Aliza Amiel, Ein-Sarid (IL); Moshe D. Fejgin, Tel-Aviv (IL)

Correspondence Address: G.E. Ehrlich (1995) Ltd., c/o Anthony Castorina, 2001 Jefferson Davis Highway, Suite 207, Arlington, VA, 22213, US

(73) Assignee: Mor Research Applications Ltd., Kyrat Matalon (IL)

(21) Appl. No.: 10/405,698

(22) Filed: Apr. 3, 2003

(51) Int. Cl. G01N 33/53; G01N 33/567

(52) U.S. Cl. 435/7.2

(57) ABSTRACT

A method is provided for determining the gender and/or chromosomal abnormality, e.g. chromosomal aneuploidy of a fetus comprising identifying fetal cells in a transcervical cell sample obtained from a pregnant woman, for example with an antibody specific to the HLA-G antigen expressed only by extravillous trophoblast cells, and then subjecting the identified fetal cells to fluorescence in situ hybridization (FISH) with one or more probes for detecting the sex of the fetus or a chromosomal abnormality such as a monosomy, a trisomy or a polyploidy, e.g. triploidy.

### BACKGROUND OF THE INVENTION

The present invention relates to prenatal genetic diagnosis and, in particular to a method of genetic diagnosis in transcervical cell (TCC) samples that permit to distinguish between maternal and female fetal cells and, thus, to detect fetus gender and chromosomal abnormality, e.g. chromosomal aneuplody, both of female and male fetuses.

Abbreviations: AEC: aminoethylcarbazole; CVB: chorionic villus biopsy; FISH: fluorescence in situ hybridization; HRP: horseradish peroxidase; IHC: immunohistochemical; mAb: monoclonal antibody; PBS: phosphate-buffered saline; PCR: polymerase-chain reaction; TCC: transcervical cell(s).

### BACKGROUND OF THE INVENTION

First trimester prenatal diagnosis of fetal abnormalities is of utmost importance. In addition to the advantage of shortening maternal anxiety, early diagnosis minimizes damages caused by potential need for intervention. Amniocentesis at 16 to 20 weeks of gestation, followed by cell culture and cytogenetic analysis, is accepted as one of the most reliable and safe methods of detecting chromosomal abnormalities. However, it carries a 0.5-1.0% risk of miscarriage. Moreover, results are rarely obtained before the 18^{th} week of pregnancy due to the time required for cell culture. Chorionic villus biopsy (CVB), although capable of providing information in the first trimester, has a 2-4% procedure-related risk of miscarriage, and may be associated with an increased risk of fetal abnormality such as defective limb development, presumably due to haemorrhage or embolism from the aspirated placental tissues (Miller et al, 1999).

When the feasibility of detecting fetal cells in the maternal circulation was first disclosed, this was seen as the future of prenatal genetic diagnosis, with the potential of replacing both amniocentesis and CVB. However, circulating fetal cells are present in the maternal blood in exceptionally low numbers and their isolation is particularly difficult, involving very expensive and labor-intensive enrichment methods. In addition, since fetal cells persist in the maternal circulation for a long time even postpartum, it may not be clear if the cells originate from the current or a previous pregnancy. This combination of problems, technical, cost and the uncertainty of the origin of the cells has prevented this approach from actually becoming a clinically acceptable one.

The presence of trophoblastic cells (shed from the placenta) in the cervical canal and their potential use for prenatal sex determination was first disclosed in the 1970's (Shettles, 1971; Rhine et al, 1975; reviewed in Holzgreve and Hahn, 2000). Four techniques have been proposed for the retrieval of cervical trophoblast cells for purpose of genetic analysis: (i) aspiration; (ii) cytobrush or cotton wool swabs; (iii) endocervical lavage; and (iv) intrauterine lavage.

The polymerase-chain reaction (PCR) technology, that revolutionized molecular diagnostics, was first used to determine fetal sex from fetal DNA retrieved from TCC trophoblast cells (either retrieved with cotton wool swabs or by flushing of the lower uterine cavity with 5 ml saline) at the beginning of the 1990's (Griffith-Jones et al, 1992). In a study in which cotton wool swabs was used, out of 26 samples taken from pregnant women at 8-13 weeks, Y chromosome-specific amplification was obtained in all but one. However, there were several false positives cases, which could be attributed to residual semen in the cervix. In all of the seven samples obtained by lavage, syncytial clumps that stained positive with the trophoblast-specific monoclonal antibodies (mAbs) NDOG1 and FT1.41.1 could be identified. Since these fetal TCC samples were contaminated by maternal cells, the authors suggested that by using fetal-specific antibodies and magnetic cell sorting, pure populations of fetal cells could be obtained. These techniques would, however, at least in part defeat the goal of providing a simple and rapid prenatal diagnostic test.

Several years later, analysis of fetal sex in TCC DNA by nested PCR was successful in samples obtained by mucus aspiration or by cytobrush. The success of fetal sex prediction was high in both methods without significant difference between the two methods of TCC sampling (Falcinelli et al, 1998).

Recently, in order to determine the presence of trophoblastic cells and to distinguish them from the predominant maternal cell population in TCC samples, the utilization of several anti-trophoblast mAbs (mAbs against placental antigens, macrophages and epithelial membrane antigens), was described (Griffith-Jones et al, 1992; Adinolfi et al, 1993; Bulmer et al, 1995). Trophoblast cells reacted with various such mAbs but displayed considerable antigenic heterogeneity, leading the authors to suggest that a panel of carefully characterized mAbs would be required to achieve a significant amount of trophoblastic cells from TCC samples.

Lately a human trophoblast HLA-G antigen specific mAb, designated G233, was introduced and shown to recognize all populations of extravillous trophoblasts (Loke et al, 1997). The HLA-G antigen recognized by mAb G233 is expressed only by extravillous trophoblast cells, and is therefore considered to be an adequate marker for circulating endovascular extravillous trophoblast cells. Recently, mAb G233 was shown to be successful in identifying fetal trophoblast cells in the maternal peripheral blood during early pregnancy. By combining immunophenotyping with the HLA-G-specific mAb G233 with detection of chromosomes 21, X, and Y, by multicolor FISH, one case of trisomy 21 was found (van Wijk et al, 2001).

As previously mentioned, four techniques have been proposed for the collection of TCC samples: (i) aspiration; (ii) cytobrush or cotton wool swabs; (iii) endocervical lavage; and (iv) intrauterine lavage. Several studies have compared these techniques and their yields. For example, cytotrophoblast cells were more abundant in TCC samples collected by lavage than in those collected by aspiration, and samples obtained by cytobrush were shown to contain more maternal cellular fragments, as a result of the abrasive procedure (Adinolfi and Sherlock, 2001).

Further investigations aimed at improving the collection techniques were prompted by the detection of nuclei containing Y fluorescent signals—when the fetus was male—and the detection of a case of trisomy 18 among the first group of mothers tested (Adinolfi et al, 1993). Thus, in subsequent studies, cytobrush, aspiration, or lavage were employed (Rodeck et al, 1995; Adinolfi et al, 1995; Massari et al, 1996; Adinolfi and Sherlock, 1997; Falcinelli et al, 1998; Miller et al, 1999). These studies were all performed on small numbers of samples collected at about 10-12 weeks of gestation, just before termination of pregnancy. The presence of cells from male fetuses was documented using FISH, conventional PCR, or quantitative fluorescent PCR (QF-PCR) assays with DNA markers specific for chromosome Y. Although various methods were used, the results clearly indicated the presence of trophoblastic cells in a large proportion of TCC samples (based on the detection of a Y chromosome in cases with male fetuses).

A variation in the percentages of TCC samples containing fetal cells was found not only according to the method employed for their collection but also according to the skill of the operator (Rodeck et al, 1995). Aspiration of the cervical mucus produced “positive” results in 50% to 70% of cases, while lavage could result in the detection of male cells in up to 80%-90% of TCC samples retrieved from mothers with male fetuses. As already mentioned, in a small group of TCC samples collected by cytobrush, all samples obtained from mothers with male fetuses were positive for chromosome Y DNA when tested by PCR, while two-thirds showed Y signals when investigated using FISH.

The present inventors have recently confirmed that the great majority (97%) of male TCC samples collected with a cytobrush contain fetal cells (Fejgin et al, 2001).

In most studies PCR and FISH assays have been utilized for the detection of aneuploidy and Y-derived DNA sequences in TCC obtained from pregnant women bearing male fetuses. The prenatal detection of X-linked or recessive inherited disorders in TCC samples of female fetuses was, until recently, impossible due to the difficulty to confirm the presence of trophoblastic cells in samples collected from mothers who carry a female fetus, because of the lack of highly polymorphic markers for the detection of paternally derived X chromosomes. One approach for the solution of this problem is the use of a pentanucleotide short tandem repeat (STR), termed X22, present on the pseudoautosomal region (PARA2) of the long arm of both X and Y chromosomes (Cirigliano et al, 1999 a, b). The detection of the paternally derived X chromosome marker in the TCC samples can be taken as clear evidence of the presence of fetal cells in the tested TCC sample.

Cioni et al, 2002, and Bussani et al, 2002, collected TCC samples by intrauterine lavage, a technique considered to be effective in yielding fetal cells, but more invasive than the other TCC sampling procedures. Analysis of the samples by FISH and PCR showed the same efficiency. With the FISH analysis, nuclei bearing X and Y signals were observed in 32/40 (Cioni et al) and 41/45 (Bussani et al) cases from known male pregnancies.

Bulmer et al, 2003, collected TCC samples by lavage. The samples were immunostained using anti-HLA-G mAb G233 and low and high molecular weight cytokeratins. The identified cells were separated using laser micro-dissection The detection rate of fetal cells using PCR was 58%, and 50% by HLA-G. Only in 4 of these identified cells, diagnosis of aneuploidy was attempted, succeeding only in two.

Cioni et al, 2003, expressed their doubts about effectiveness and reliability of the transcervical approach for prenatal diagnosis. They collected TCC samples by cytobrush and submitted the samples to PCR analysis. Placental tissue samples were analysed by FISH for fetal sexing. In their hands, fetal cells were not detected in a constant and reliable fashion in cervical mucus samples collected in the first trimester of pregnancy. Fetal cells could be detected in 19 to 33% of male samples. A disappointing 0.5 to 2% rate of fetal cells in only 3 out of 13 cases from male pregnancies was found. Both the FISH and the PCR approaches did not allow discrimination between maternal and female fetal cells within a sample. The authors therefore concluded that mucus sampling can yield fetal cells in a limited number of cases and cannot therefore be regarded as a tool of clinical value for the attainment of minimally invasive prenatal diagnosis.

Early, non-invasive prenatal diagnosis remains an ideal but not as yet accomplished approach. It would be of great importance to develop an efficient, simple and non-invasive method of prenatal genetic diagnosis that could be carried out early in the first trimester of the pregnancy, thus allowing the parents to reach a decision about the fate of the pregnancy in privacy and before emotional bonding occurs.

### SUMMARY OF THE INVENTION

It has now been found, in accordance with the present invention, that by combination of the use of HLA-G specific antibody in order to identify fetal cells in TCC samples and application of the FISH technique to the thus identified fetal cells, the fetus gender and chromosomal abnormality, e.g. chromosomal aneuploidy, can be detected.

The present invention thus provides a method for determining the gender and/or chromosomal abnormality, e.g. chromosomal aneuploidy, of a fetus, consisting essentially of the steps:

(i) obtaining a maternal transcervical cell sample;

(ii) cytospining a suspension containing said cell sample;

(iii) spreading the cells obtained in (ii) on a slide

(iv) subjecting the cells on the slide to immunohistochemical staining using an antibody specific for the identification of fetal cells;

(v) analyzing the cells of step (iv) under a microscope and identifying the fetal cells recognized by said antibody;

(vi) evaluating the identified fetal cells of (v) by fluorescence in situ hybridization (FISH) to determine fetal gender and/or chromosomal abnormality, e.g. chromosomal aneuploidy.

Preferably, the transcervical cell sample is obtained by cytobrush, i.e a cytobrush smear, and the antibody used for identification of the fetal cells is an HLA-G specific antibody.

### BRIEF DESCRIPTION OF THE FIGURES

FIG. 1 shows FISH of cells obtained by cervical cytobrushing of a pregnant woman at the 9^{th} week of gestation (case 13 in Table 1). A male fetus cell is identified by the red (chromosome Y) and green (chromosome X) signals. Residues of the HLA-G antibody used in the immunohistochemical assay carried out before FISH can be seen at the lower part of the cell (red).

FIGS. 2A-2B show immunohistochemistry and FISH, respectively, of cells obtained by cervical cytobrushing of a pregnant woman at the 8^{th} week of gestation (case 66 in Table 1). FIG. 2A depicts a cell (arrow) showing the immunohistochemistry with the HLA-G antibody. The antigen-antibody reaction appears as a halo around the cell stained brick red. FIG. 2B depicts the same cell (arrow) after FISH, with one red signal that represents the single X chromosome present in the female cell (indication of Turner's syndrome).

FIGS. 3A-3B show immunohistochemistry and FISH, respectively, of cells obtained by cervical cytobrushing of a pregnant woman at the 12^{th} week of gestation (case 30 in Table 1). FIG. 3A depicts a cell (arrow) showing the immunohistochemistry with the HLA-G antibody. The antigen-antibody reaction appears as a halo around the cell stained brick red. FIG. 3B depicts the same cell (arrow) after FISH, with three signals (green) of chromosome 18. The three copies of chromosome 18 represent trisomy 18 (Edward's syndrome).

FIG. 4 is a graph showing the percentage of fetal cells identified by immunohistochemistry with the HLA-G antibody (Horizontal lines) and the percentage of fetal cells confirmed by FISH (vertical lines).

### DETAILED DESCRIPTION OF THE INVENTION

The present invention provides a method for determining the gender and/or chromosomal abnormality or aberration, e.g. chromosomal aneuploidy of a fetus in a maternal transcervical cell sample from a pregnant woman at the 6^{th} to 14^{th} week of gestation. The sample may be obtained by any suitable method, but is preferably carried out by cytobrush, and is more preferably a cytobrush smear. Thus, contrary to the invasive amniocentesis and chorionic villus biopsy techniques currently in use, that have to be carried out in hospitals, the cells to be examined according to the method of the present invention can be removed by a gynecologist or other health care provider in private or ambulatory cliniques by brushing or scraping the cervix using standard tools, e.g. speculum. The sample thus obtained is shaken in an appropriate medium and is then sent to the laboratory for genetic examination.

The maternal and fetal cells contained in the sample are obtained from the cell suspension by cytospining the suspension, and are then immobilized, for example by spreading on a glass slide.

In order to identify the fetal cells and distinguish them from the maternal cells, the cells spread on the slide are subjected to immunohistochemical staining using an antibody specific for the identification of fetal cells.

Any antibody that specifically binds a fetal cell antigen, but not a maternal cell antigen, can be used according to the invention to characterize the fetal cell. This antibody is sometimes referred to herein by the term “primary antibody”. In a preferred embodiment, the primary antibody is an antibody specific to the HLA-G antigen that is expressed only by extravillous trophoblast cells. Several anti-HLA-G monoclonal antibodies are commercially available.

In one step of the invention, the cells of the sample spread on the slide are contacted with the primary antibody, preferably the antibody that is specific for the HLA-G antigen, so as to allow the antibody to bind the antigen. According to normal protocols, following antibody binding to the sample, any unbound antibody is removed in a wash step, e.g., with PBS (phosphate-buffered saline) or Tris-based buffer with or without non-ionic detergent.

In one embodiment of the invention, the primary antibody is detected using a “secondary antibody” (i.e., an anti-antibody antibody such as a goat anti-mouse IgG antibody) which is itself labeled, for example with biotin, or otherwise detectable.

The antibody binding may be detected directly or indirectly, for example using an enzymatic reporter molecule such as, but not limited to, a phosphatase such as alkaline phosphatase or a peroxidase such as horseradish peroxidase (HRP), that acts on a substrate to produce a detectable product to detect the antibody. When the secondary antibody is labeled with biotin, the enzymatic reporter molecule is conjugated with avidin or streptavidin. In one preferred embodiment, the secondary antibody is a biotinylated anti-anti-HLA-G antibody antibody, the enzymatic reporter molecule is HRP-streptavidin conjugate, and the HRP substrate is aminoethylcarbazole (AEC). In the presence of H₂O₂, peroxidases catalyze the oxidation of AEC into a brick red precipitate and, thus, the fetal cells recognized by the primary anti-HLA-G antibody are brick red stained. The cells are further stained with hematoxylin, that stains the nucleus blue.

After the immunohistochemical staining described above, the slide containing the cells is washed, covered with a mounting solution, a cover glass is put on the slide, and the cells are scanned using a light microscope. The analysis of the cells under the microscope may be made in conjunction with an automated image analysis system. Each stained cell, namely each identified fetal cell, is located with its coordination numbers in the microscope. In addition, the image of the cell can be preserved by special computer programs for further comparison.

In the next step, the slides containing the stained cells are subjected to FISH, namely to hybridization of a fluorescent polynucleotide probe or probes capable of specifically annealing to a DNA or RNA sequence in a cellular nucleic acid and detection of the resulting hybrid. Briefly, conventional in situ hybridization assays generally comprises one or more of the following steps: (1) prehybridization treatment of the biological structure to increase accessibility of target DNA or RNA (e.g., denaturation with heat or alkali); (2) optionally (and depending on the probe) steps to reduce nonspecific binding (e.g., by blocking the hybridization capacity of repetitive sequences, e.g., using human genomic DNA); (3) hybridization of one or more nucleic acid probes to the nucleic acid in the biological structure or tissue; (4) post-hybridization washes and/or nuclease digestion to remove nucleic acid fragments not bound in the hybridization; and, (5) detection of the hybridized nucleic acid fragments. The reagents used in each of these steps and conditions for their use vary depending on the particular application.

According to the present invention, the FISH step is used to detect a chromosomal DNA of the identified fetal cells. In one preferred embodiment, the detectably-labeled probe is hybridized to a chromosomal sample to detect karyotype (e.g., the presence of a Y or X chromosome), chromosome aberrations or chromosome number (e.g., sex chromosomal trisomies, XXY, XYY etc., resulting in 47 chromosomes).

For the FISH procedure, chromosome-specific probes are used that consist in a combination of detectably labeled polynucleotides that have sequences corresponding to (e.g., essentially the same as) the sequences of DNA from a particular chromosome or subchromosomal region of a particular chromosome (e.g., a chromosome arm). Typically, the chromosome-specific probe is produced by amplification (e.g., using the polymerase-chain reaction) of the corresponding chromosomal DNA. A chromosome-specific probe will hybridize in an essentially uniform pattern along the chromosome or subchromosomal region from which it is derived. Many chromosome probes or chromosome paints labeled with fluorescent labels are commercially available, for example probes labeled with SpectrumOrange™, SpectrumGreen™ and SpectrumAqua™ of Vysis Inc. (Downers Grove, Ill., USA). In a preferred procedure, FISH can be carried out with a cocktail of probes, for example a cocktail of probes for X and Y chromosomes, or for X, Y and 21 chromosomes, or for X, 13 and 18 chromosomes,

The probes used according to the invention may be probes for determination of the fetus gender, namely probe sets used for hybridization to the Y chromosome and/or to the X chromosome. These chromosome probes or chromosome paints are commercially available, including probes for hybridization to both the X and Y chromosomes.

The probes may also be useful to detect chromosomal aberration or chromosome abnormality, referring to a deviation between the structure of the subject chromosome or karyotype and a normal (i.e., “non-aberrant”) homologous chromosome or karyotype. The terms “normal” or “non-aberrant,” when referring to chromosomes or karyotypes, refer to the predominant karyotype or banding pattern found in healthy individuals of a particular species and gender. Chromosome abnormalities or aberrations can be numerical or structural in nature, and include aneuploidy, i.e. any deviation from an exact multiple of the haploid number of chromosomes, whether fewer (hypoploidy as in Turner's syndrome) or more (hyperploidy or polyploidy), or inversion (alteration in sequence), translocation (exchange), deletion (loss), or duplication (gain), of genetic material. Chromosome abnormalities may be correlated with the presence of a pathological condition, e.g., trisomy 21 in Down syndrome, trisomy 18 in trisomy 18 or Edward's syndrome, and trisomy 13 in trisomy 13 or Patau's syndrome. These chromosome probes or chromosome paints are commercially available.

When cells are screened microscopically, it is sometimes desirable to counterstain the cells, using a stain or dye that is of a contrasting color to a specific stain or dye used to identify a particular target, e.g., to enhance detection of nuclei. For FISH, counterstains consisting of fluorophores are used to stain DNA a contrasting color to distinguish the background DNA from the specifically labeled probe hybridized to the target DNA. A suitable counterstain is DAPI (4,6-diamidino-2-phenylindole), a DNA dye which fluoresces (“blue”) when exposed to ultraviolet light and is used to stain the nuclear or background DNA of the cell in FISH assays. DAPI is the nucleus counterstain used when the probe used to mark the desired chromosome is labeled with SpectrumOrange, SpectrumGreen, or an FITC-based fluorescent dye.

According to the invention, in the last step, after FISH with the chosen probe, the fetal cells are evaluated under the microscope to determine fetal gender and/or chromosomal aberration, e.g. chromosome aneuploidy such as trisomy 21, trisomy 13 and/or trisomy 18, monosomy as in Turner's syndrome, or triploidy.

In another embodiment, the present invention provides a method for determining the sex of a fetus, comprising the steps:

(i) obtaining a sample of transcervical cells from a woman pregnant with a fetus;

(ii) spreading the cells from said sample on a slide;

(iii) subjecting the cells on the slide to immunohistochemical staining using an antibody specific for the identification of fetal cells;

(iv) analyzing the cells of step (iii) under a microscope and identifying the fetal cells recognized by said antibody;

(v) treating the slide containing the identified fetal cells such that fetal DNA present in fetal nucleated cells present in the sample is made available for hybridization resulting in available fetal DNA;

(vi) evaluating the cells by fluorescence in situ hybridization (FISH) by contacting the available fetal DNA with a fluorescent DNA probe hybridizable to fetal Y or X chromosomal DNA under hybridization conditions; and

(vii) detecting the presence of hybridization between the fluorescent DNA probe and the fetal Y or X chromosomal DNA, the hybridization with the Y and the X chromosomal DNA being an indication of a male fetus and the hybridization with two of the X chromosomal DNA being an indication of a female fetus.

In a further embodiment, the present invention provides a method for detecting a chromosomal abnormality or aberration in a fetus, comprising:

(i) obtaining a sample of transcervical cells from a woman pregnant with a fetus;

(ii) spreading the cells from said sample on a slide;

(iii) subjecting the cells on the slide to immunohistochemical staining using an antibody specific for the identification of fetal cells;

(iv) analyzing the cells of step (iii) under a microscope and identifying the fetal cells recognized by said antibody;

(v) treating the slide containing the identified fetal cells such that fetal DNA present in fetal nucleated cells present in the sample is made available for hybridization resulting in available fetal DNA;

(vi) evaluating the cells by fluorescence in situ hybridization (FISH) by contacting the available fetal nucleated DNA with a fluorescent DNA probe hybridizable to a chromosomal fetal DNA associated with a chromosomal abnormality of interest, under hybridization conditions; and

(vii) detecting the presence or absence of hybridization between the fluorescent DNA probe and the chromosomal fetal DNA of interest as an indication of the presence or absence of said chromosomal abnormality or aberration.

The present invention further relates to a kit for carrying out the method of the invention, the kit including, packaged together in a container, one or more of the following reagents: (a) a primary antibody specific to HLA-G antigen; (b) a labeled, e,g, biotinylated, secondary antibody; (c) a HRP-avidin or streptavidin conjugate; (d) a HRP substrate, e.g. AEC; and (e) at least one chromosome-specific fluorescent probe for FISH, for example for chromosome X, Y, 13, 18 or 21.

The present invention provides a very reliable and effective tool for early non-invasive prenatal genetic diagnosis. The method of the invention can be used alone or together with known invasive methods when fetal cells cannot be identified by the method.

The invention will now be illustrated by the following non-limitative examples.

### EXAMPLES

Materials and Methods

(i) Patients and Sampling Procedure

Sixty-eight pregnant women between 6 and 14 weeks of gestation, who were scheduled to undergo pregnancy termination, were enrolled in this study. After giving their informed consent, the patients were taken to the operating room and put in the lithotomy position. A Pap smear cytobrush (MedScand-AB, Malmo, Sweden) was inserted through the external os to a maximum depth of 2 cm (the brush's length), and removed while rotating it a full turn. The material that was caught on the brush was shaken into a test tube that contained 2-3 ml medium M-199+1%+antibiotics. Cytospin preparations from all samples were kept in 95% alcohol until the commencement of the laboratory experiments. The cells were spread on slides for analysis by FISH.

The pregnancy was then terminated by suction curettage. A piece of placental tissue was then removed and taken for FISH procedure.

In selected cases (when FISH identified a male pregnancy and/or aneuploidy), immunohistochemical (IHC) procedure was applied to the material obtained from the cervix by smear cytobrush. After identifying fetal cells, the coordinations were recorded. The dye was removed from the slide. FISH was applied to the same slides, and read according to the recorded location of the fetal cells.

(ii) Preparation of Placental Tissue

The following protocol was followed in the preparation of placental tissue as a control in order to identify the sex and chromosomal aneuploidy of the fetus.

Placental tissue obtained after termination of pregnancy was squashed with a scalpel. The tissue was washed with buffer containing KCl and sodium citrate (1:1), the fluid was flushed with a pipette, incubated at room temperature for 13 min, added to a fixer (3:1 solution of methanol:acetic acid), and incubated for another 45 min at room temperature. This procedure was repeated 1-3 times. Then, the fluid was flushed, 3 drops of acetic acid 6% were added thereto, and the mixture was transferred onto slides. The slides were dried at room temperature and sent to the genetic laboratory for analysis by FISH.

(iii) Immunohistochemical (IHC) Staining Protocol

The slides containing the cells were washed in 70% alcohol solution, removed to distilled water (DW) for 5 min and then moved into a moist chamber, and washed (3 times) with PBS. The borders of the cytobrush smear cells were marked with PaP Pen (Zymed Laboratories Inc., San Francisco, Calif., USA). After incubation with 50 μl peroxide 3% for 10 min at room temperature, the slides were washed (3 times) with PBS, shaken, 2 drops of blocking reagent (Zymed) were added, followed by incubation for 10 min. The primary HLA-G specific antibody (mAb 7758, Abcam Ltd., Cambridge, UK), diluted 1:200 in antibody diluent reagent solution (Zymed), was added (40 μl to each slide), the slides were incubated in moist chamber for 60 min, and washed (3 times) with PBS. Then, a biotinylated compatible secondary antibody (goat anti-mouse IgG antibody, Zymed Cat No. 858943), was added (2 drops to each slide), the slides were incubated in moist chamber for 15 min, washed (3 times) with PBS and shaken. Following addition of horseradish peroxidase (HRP)-streptavidin conjugate (2 drops to each slide; Zymed), the slides were incubated in moist chamber for 15 min, washed (3 times) with PBS, 2 drops (to each slide) of a solution of HRP substrate aminoethylcarbazole (AEC Single Solution Chromogen/Substrate, Zymed) were added, the slides were incubated in moist chamber for 10 min and washed (3 times) with PBS. The cytobrush smear cells were stained with hematoxylin (that stains the nucleus blue) for 25 sec, washed with tap water, the tissue section was covered with glycerol vinyl alcohol (GVA) aqueous mounting solution (Zymed), and the cover glass put on each slide. The slides with the cytobrush smear were scanned using a light microscope and the location of the stained cells (trophoblasts) was marked with its coordination numbers in the microscope. In this assay, a kit was used that contains the blocking solution, the biotinylated secondary antibody, the HRP-streptavidin conjugate and the AEC solution (Zymed HISTOSTAIN®-PLUS Kit, Cat No. 858943).

(iv) Preparation of IHC Slides for the FISH Technique

FISH was applied to the same slides of (iii), after removal of the dye. The slides were put in double-distilled water (DDW) for 20 min, dehydrated in a graded ethanol series (50%, 70%, 100%), added to a fixer solution (3:1 solution of methanol: acetic acid) for 10 min, put into a 100% methanol solution for 10 min, and dried at room temperature.

(v) Fluorescence In Situ Hybridization (FISH), Direct Preparation

The slide spreads were denatured for 2 min in 70% formamide/2×SSC at 70° C. and dehydrated in a graded ethanol series. The probe mix was then applied to air-warmed slides (30 ml mix sealed under a 24×50 mm glass coverslip) and hybridized for 18 h at 37° C. in a moist chamber. Following hybridization, the slides were washed in 50% formamide/2×SSC for 20 min at 430, rinsed in two changes of 2×SSC at 37° for 4 min each, and placed in 0.05% Tween 20 (Sigma, Israel). The slides were counterstained in DAPI (Sigma, Israel) antifade solution and analyzed for simultaneous viewing of FITC, Texas-Red and DAPI.

For the FISH analysis, a two-color technique was carried out with the following direct-labeled probes supplied by Vysis Inc. (Downers Grove, Ill., USA):

(1) For identifying sex chromosome, the probe CEP®X SpectrumGreen™/CEP® Y (a satellite) SpectrumOrange™ (Vysis Cat. No. 32-131051), CEP X/Y consists of a satellite DNA specific to the centromere region Xp11.1-q11.1 (DXZ1) directly labeled with SpectrumGreen™ and mixed with probe specific to a satellite DNA sequences contained within the centromere region Yp11.1-q11.1 (DYZ3) directly labeled with SpectrumOrange™.

(2) For chromosome 21, for identifying trisomy 21, responsible for the Down syndrome phenotype, the probe LSI® 21 SpectrumOrange™ (Vysis Cat No. 32-190002). LSI 21 contains unique DNA sequences complemenrary to the loci D21 S259, D21 S341 and D21 S342 contained within the 21 q22.13 to 21 q22.2 region on the long arm of chromosome 21. This probe is used to determine the copy number of chromosome 21 in interphase and metaphase cells.

(3) For chromosome 13, for identifying trisomy 13, the probe LSI® 13 (13q14) SpectrumGreen™ (Vysis Cat No. 32-192018). LSI 13 contains unique DNA sequences specific to the 13q14 region of chromosome 13. LSI 13 can be used to determine the copy number of chromosome 13 in interphase and metaphase cells.

(4) For chromosome 18, for identifying trisomy 18, the probe CEP®18 (D18Z1, α satellite) SpectrumGreen™ (Vysis Cat No. 32-130048). The CEP 18 probe consists of DNA sequences specific to the alpha satellite DNA (D18Z1) contained within the centromeric region (18p 11.1-q 11.1) of chromosome 18.

(vi) FISH Analysis

FISH was applied to the IHC identified fetal cells. In cases in which the dye was washed off, the cells were identified according to their known location on the slide, as marked according to the microscope coordinates.

Results:

The transcervical cells obtained from the pregnant women were analyzed only in cases in which FISH of the placental tissue revealed a male fetus and/or a chromosomal aberration. Sixty-eight (68) such cases (male and abnormal karyotype) were included in the study.

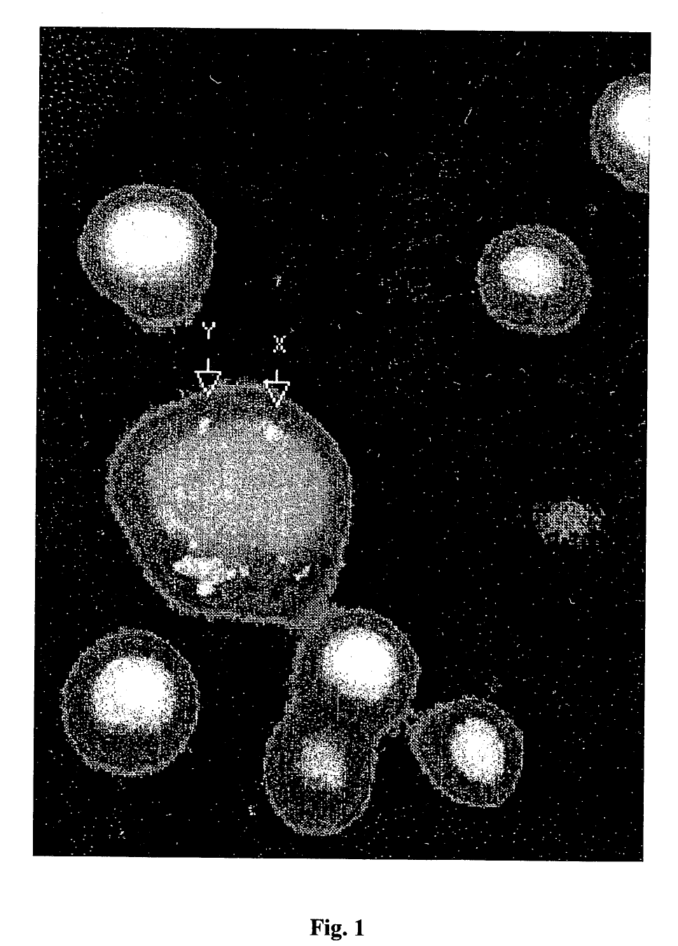

The results are shown in Table 1 and in FIGS. 1-4.

Table 1 shows the gestational age at the time of the sampling, the number of cells marked by IHC (fetal cells identified by the HLA-G specific mAb) and the confirmation of cells marked by FISH.

As shown in Table 1 and in FIG. 4, from the 68 cases, fetal cells were detected in 63 cases (in Table 1, number of cells marked by IHC is higher than 0; about 93%, as shown in FIG. 4). In 47 of the 63 cases with fetal cells (in Table 1, number of cells marked by FISH is higher than 0; about 75%, as shown in FIG. 4), was FISH successful.

As can be seen, both male and female fetuses were correctly identified.

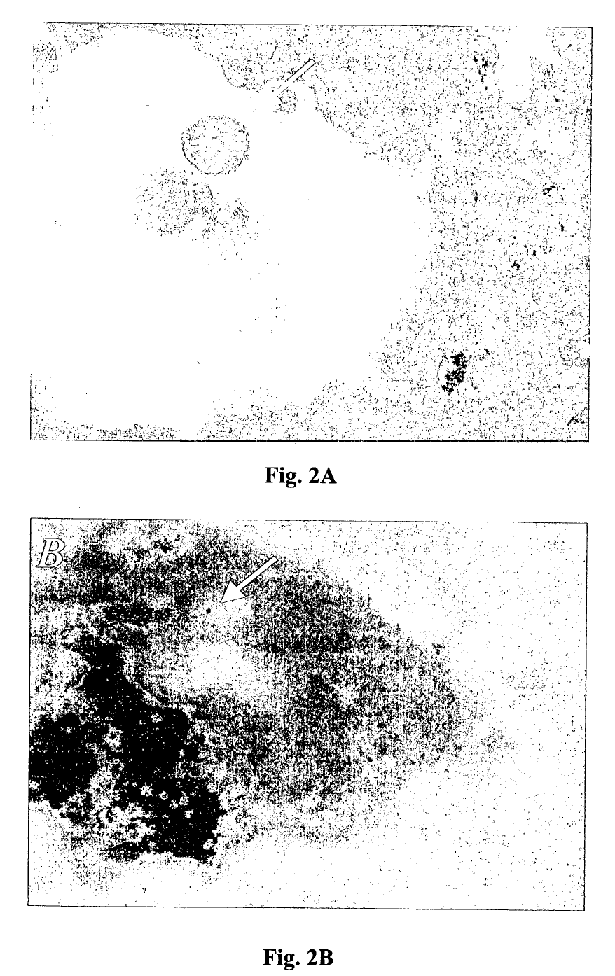

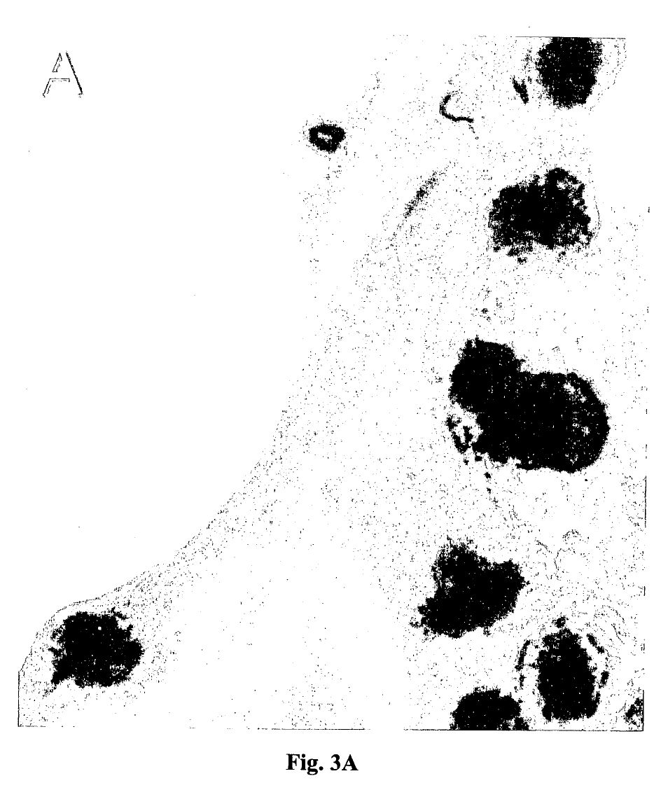

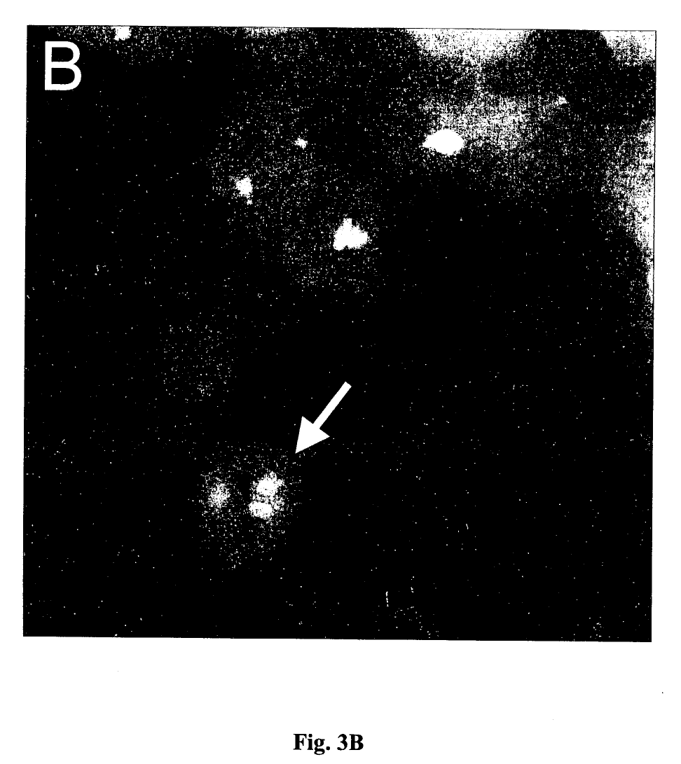

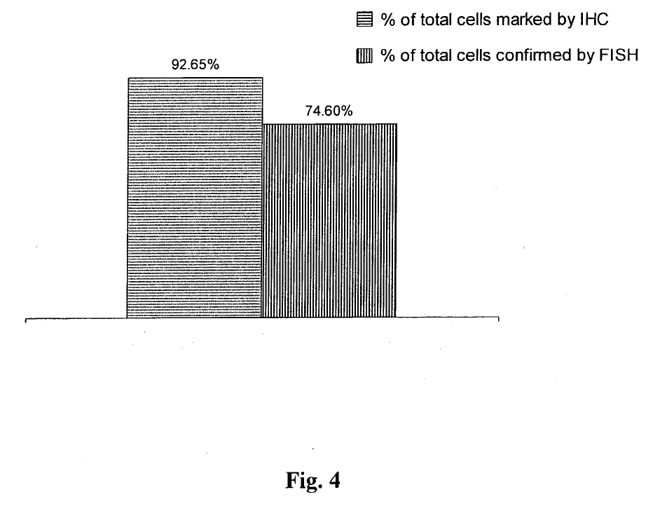

Several cases of chromosomal aberration were detected. In 43 of the 47 cases marked by FISH (91.5%), gender and/or aberration was correctly diagnosed. Most chromosomal aberrations (26/30, 87%) were detected in the cases with missed abortions. The 3 cases of 45,XO were correctly identified. Out of the 8 cases of Trisomy 21, seven were correctly identified (in the remaining case FISH was not successful). There was one case of Trisomy 18 and it was correctly identified (FIG. 3). Out of the 3 cases of Trisomy 13, in two was FISH successful. The correct diagnosis was established only in one of the two cases (one case was false negative). FIG. 1 shows a cell of a male fetus without aberration (case 13 in Table 1). FIG. 2 shows a case of monosomy X (Turner's syndrome; case 66, XO). FIG. 3 shows a case of trisomy 18 (Edward's syndrome).

Triploidy was identified when two sets of different trisomies appeared in the same cell.

Summarizing the results: When FISH was successful, sex determination was correct in 29 out of 30 cases analyzed (Table 1). Aneuploidy was detected in 17 of 21 cases following successful FISH (Table 1).

TABLE 1
Identifying fetal cells with HLA-G antibody and
confirmation with FISH.

| | | | No. | | | |
|---|---|---|---|---|---|---|
| | | No. | cells | | Aber- | |
| | | cells | marked | | rations | XY/ |
| Case | Gest. | marked | by | Gender | iden- | aberrations |
| No. | Weeks | by IHC | FISH | placenta | tification | identification |
| 1 | 12 | 3 | 0 | XY/XXY | | − |
| 2 | 10 | 14 | 3 | XY | | + |
| 3 | 8 | 12 | 6 | XY | | + |
| 4 | 11 | 2 | 2 | XY | | + |
| 5 | 8 | 8 | 5 | XY | | + |
| 6 | 10 | 7 | 2 | XY | | + |
| 7 | 8.5 | 1 | 0 | XY | | − |
| 8 | 10 | 1 | 1 | XY | | + |
| 9 | 11 | 2 | 0 | XY | | false |
| 10 | 9.5 | 11 | 1 | XY | | + |
| 11 | 12 | 4 | 0 | XY | | − |
| 12 | 7.5 | 10 | 2 | XY | | + |
| 13 | 9 | 10 | 4 | XY | | + |
| 14 | 9.5 | 4 | 3 | XY | | + |
| 15 | 9 | 5 | 1 | XY | | + |
| 16 | 8 | 6 | 2 | XY | | + |
| 17 | 6 | 0 | 0 | XY | | − |
| 18 | 6 | 1 | 0 | XY | | − |
| 19 | 5 | 1 | 0 | XY | | − |
| 20 | 7 | 2 | 1 | XY | | + |
| 21 | 9.5 | 3 | 2 | XY | | + |
| 22 | 9 | 4 | 1 | XY | | + |
| 23 | 9 | 12 | 4 | XY | | + |
| 24 | 7 | 9 | 1 | XY | | + |
| 25 | 10 | 1 | 0 | XY | | − |
| 26 | 7 | 4 | 2 | XY | | + |
| 27 | 14 | 1 | 0 | XY | | − |
| 28 | 9 | 1 | 0 | XY | | − |
| 29 | 9 | 0 | 0 | XY | | − |
| 30 | 12 | 31 | 17 | XY | 3 × 18 | + |
| 31 | 9 | 7 | 0 | XY | 3 × 13 | − |
| 32 | 9 | 26 | 12 | XY | | + |
| 33 | 7 | 4 | 2 | XY | | + |
| 34 | 10 | 7 | 6 | XY | 3 × 21 | + |
| 35 | 7 | 7 | 0 | XY | | − |
| 36 | 8.5 | 18 | 7 | XY | | + |
| 37 | 8 | 22 | 6 | XY | | + |
| 38 | 7 | 10 | 10 | XY | | + |
| 39 | 7 | 9 | 2 | XY | | + |
| 40 | 9 | 4 | 2 | XY | | + |
| 41 | 10 | 8 | 2 | XY | | + |
| 42 | 9.5 | 2 | 1 | XY | | + |
| 43 | 8 | 6 | 2 | XXYY | | + |
| 44 | 8 | 0 | 0 | XXY | Triploidy | − |
| 45 | 10.5 | 13 | 5 | XXY | Triploidy | + |
| 46 | 9 | 4 | 0 | XXY | XXY | − |
| 47 | 13 | 9 | 5 | XXX | Triploidy | false |
| 48 | 13 | 0 | 0 | XXX | Triploidy | − |
| 49 | 6 | 0 | 0 | XXX | 3 × X | false |
| 50 | 8 | 1 | 0 | XXX | 3 × X | − |
| 51 | 10 | 3 | 1 | XXX | 3 × X | + |
| 52 | 8 | 3 | 1 | XXX | Triploidy | + |
| 53 | 7 | 3 | 0 | XXX | | − |
| 54 | 8 | 5 | 3 | XXX | | + |
| 55 | 6 | 3 | 2 | XX | 3 × 21 | + |
| 56 | 8 | 6 | 0 | XX | 3 × 13 | false |
| 57 | 7.5 | 5 | 2 | XX | 3 × 21 | + |
| 58 | 10 | 9 | 1 | XX | 3 × 21 | + |
| 59 | 12 | 8 | 3 | XX | 3 × 21 | + |
| 60 | 8 | 9 | 7 | XX | 3 × 21 | + |
| 61 | 10 | 5 | 1 | XX | 3 × 13 | + |
| 62 | 10 | 10 | 5 | XX | 3 × 21 | + |
| 63 | 9 | 2 | 0 | XX | 3 × 21 | − |
| 64 | 8.5 | 21 | 15 | X0 | | + |
| 65 | 10 | 10 | 8 | X0 | | + |
| 66 | 8 | 13 | 9 | X0 | | + |
| 67 | 8.5 | 4 | 2 | X0 | | + |
| 68 | 11 | 7 | 2 | X0 | | + |

### REFERENCES

Adinolfi M, Davies A, Sharif S, Soothill P, Rodeck C (1993) Detection of trisomy 18 and Y-derived sequences in fetal nucleated cell obtained by transcervical flushing. Lancet 342:403-404.

Adinolfi M, Sherlock J, Tutschek B, Halder A, Delhanty J, Rodeck C (1995) Detection of fetal cells in transcervical samples and prenatal diagnosis of chromosomal abnormalities. Prenat Diagn 15:943-949.

Adinolfi M and Sherlock Jon (1997) First trimester prenatal diagnosis using transcervical cells: an evaluation. Hum. Reprod. 3:383-392.

Adinolfi M, Sherlock Jon (2001) Fetal cells in transcervical samples at an early stage of gestation. J hum Genet 46:99-104.

Bulmer J N, Rodeck C, Adinolfi M (1995) Immunohistochemical characterization of cells retrieved by transcervical sampling in early pregnancy. Prenat Diagn 15:1143-1153.

Bulmer J N, Cioni R, Bussani C, Cirigliano V, Sole F, Costa C, Garcia P, Adinolfi M (2003) HLA-G positive trophoblastic cells in transcervical samples and their isolation and analysis by laser microdissection and QF-PCR. Prenat Diagn 23:34-39.

Bussani C, Cioni R, Scarselli B, Barciulli F, Bucciantini S, Simi P, Fogli A, Scarselli G (2002) Strategies for the isolation and detection of fetal cells in transcervical samples. Prenat Diagn 22:1098-1101.

Cioni R, Bussani C, Scarselli B, Barciulli F, Bucciantini S, Simi P, Fogli A, Scarselli G (2002) Detection of fetal cells in intrauterine lavage samples colleted in the first trimester of pregnancy. Prenat Diagn 22:52-55.

Cioni R, Bussani C, Scarselli B, Bucciantini S, Barciulli F, Scarselli G (2003) Fetal cells in cervical mucus in the first trimester of pregnancy. Prenat Diagn 23:168-171.

Cirigliano V, Sherlock J, Conway G, Quilter C, Rodeck C, Adinolfi M (1999a) Rapid detection of chromosomes X and Y aneuploidies by quantitative fluorescent PCR. Prenat Diagn 19:1099-1103.

Cirigliano V, Sherlock J, Petru M, Ward R T H, Rodeck C, Adinolfi M (1999b) Transcervical cells and the prenatal diagnosis of haemoglobin (Hb) mutations. Clin Genet 56:357-361.

Falcinelli C, Battafarano S, Neri C et al (1998) Analysis of fetal sex in TCC sample DNA: a contribution to the validation of this approach. Prenat Diagn 18:1109-1116.

Fejgin M D, Diukman R, Cotton Y, Weinstein G, Amiel A (2001). Fetal cells in the uterine cervix: a source for early non-invasive prenatal diagnosis. Prenat Diagn 21:619-621.

Griffith-Jones M D, Miller D, Lilford R J et al (1992). Detection of fetal DNA in trans-cervical swabs from first trimester pregnancies by gene amplification: a new route to prenatal diagnosis ? British J Obstet and Gynaecol 99:508-511.

Holzgreve W, Hahn S (2000). Fetal cells in cervical mucus and maternal blood. Clin Obstet and Gynaecol 14:709-722.

Loke Y W, King A, et al. (1997). Evaluation of trophoblast HLA-G Antigen with a specific monoclonal antibody. Tissue Antigen 50: 135-146.

Massari A, Novelli G, et al. (1996). Non-invasive early prenatal molecular diagnosis using retrieved transcervical trophoblast cells. Hum Genet 97:150-155.

Miller D, Briggs J, Rahman M S, Griffith-Jones M, Rane V, Everett M, Lilford RJ, Bulmer J N (1999). Transcervical recovery of fetal cells from the lower uterine pole: reliability of recovery and histological/immunocytochemical analysis of recovered cell populations. Human Reproduction 2:521-531.

Rhine S A, Cain J L, Cleary R E et al (1975). Prenatal sex detection with endocervical smears: successful results utilizing Y-body fluorescence. Am J Obstet Gynecol 122:155-160.

Rodeck C, Tutschek B, Kingdom J, Sherlock J (1995). Methods for the collection of transcervical samples during the first trimester of gestation. Pren Diagn 15:933-942.

Shettles L B (1971). Use of the Y chromosome in prenatal sex determination. Nature London 230:52-53.

Van Wijk I J, Griffloen S, Tjoa M L, Mulders M A M et al. (2001). HLA-G expression in trophoblast cells circulating in maternal peripheral blood during early pregnancy. Am J Obstet Gynecol 184:991-997.

1. A method for determining the gender and/or chromosomal aneuploidy of a fetus consisting essentially of the steps:
(i) obtaining a maternal transcervical cell sample;
(ii) cytospining a suspension containing said cell sample;
(iii) spreading the cells obtained in (ii) on a slide
(iv) subjecting the cells on the slide to immunohistochemical staining using an antibody specific for the identification of fetal cells;
(v) analyzing the cells of step (iv) under a microscope and identifying the fetal cells recognized by said antibody;
(vi) evaluating the identified fetal cells by fluorescence in situ hybridization (FISH) to determine fetal gender and/or chromosomal aneuploidy.

2. The method according to claim 1 wherein said transcervical cell sample is obtained by cytobrush.

3. The method according to claim 1 wherein said transcervical cell sample is obtained from a pregnant woman at the 6^{th} to 14^{th} week of gestation.

4. The method according to claim 1 wherein the antibody in step (iv) is an antibody specific to the HLA-G antigen expressed only by extravillous trophoblast cells.

5. The method according to claim 1 wherein the identified fetal cells are evaluated by fluorescence in situ hybridization (FISH) to determine fetal gender.

6. The method according to claim 5 wherein the identified fetal cells are evaluated by fluorescence in situ hybridization (FISH) to determine a female fetus.

7. The method according to claim 5 wherein the identified fetal cells are evaluated by fluorescence in situ hybridization (FISH) to determine a male fetus.

8. The method according to claim 5 wherein the identified fetal cells are evaluated by fluorescence in situ hybridization (FISH) to determine a chromosomal aneuploidy.

9. The method of claim 8 wherein the chromosomal aneuploidy is trisomy 21.

10. The method of claim 8 wherein the chromosomal aneuploidy is trisomy 18.

11. The method of claim 8 wherein the chromosomal aneuploidy is trisomy 13.

12. The method of claim 8 wherein the chromosomal aneuploidy is triploidy.

13. A method for determining the sex of a fetus, comprising the steps:
(i) obtaining a sample of transcervical cells from a woman pregnant with a fetus;
(ii) spreading the cells from said sample on a slide;
(iii) subjecting the cells on the slide to immunohistochemical staining using an antibody specific for the identification of fetal cells;
(iv) analyzing the cells of step (iii) under a microscope and identifying the fetal cells recognized by said antibody;
(v) treating the slide containing the identified fetal cells such that fetal DNA present in fetal nucleated cells present in the sample is made available for hybridization resulting in available fetal DNA;
(vi) evaluating the cells by fluorescence in situ hybridization (FISH) by contacting the available fetal DNA with a fluorescent DNA probe hybridizable to fetal Y or X chromosomal DNA under hybridization conditions; and
(vii) detecting the presence of hybridization between the fluorescent DNA probe and the fetal Y or X chromosomal DNA, the hybridization with the Y and X chromosomal DNA being an indication of a male fetus and the hybridization with two of the X chromosomal DNA being an indication of a female fetus.

14. The method according to claim 13 wherein said transcervical cell sample is obtained by cytobrush.

15. The method according to claim 13 wherein said transcervical cell sample is obtained from a pregnant woman at the 6^{th} to 14^{th} week of gestation.

16. The method according to claim 13 wherein the antibody in step (iv) is an antibody specific to the HLA-G antigen expressed only by extravillous trophoblast cells.

17. A method for detecting a chromosomal abnormality in a fetus, comprising:
(i) obtaining a sample of transcervical cells from a woman pregnant with a fetus;
(ii) spreading the cells from said sample on a slide;
(iii) subjecting the cells on the slide to immunohistochemical staining using an antibody specific for the identification of fetal cells;
(iv) analyzing the cells of step (iii) under a microscope and identifying the fetal cells recognized by said antibody;
(v) treating the slide containing the identified fetal cells such that fetal DNA present in fetal nucleated cells present in the sample is made available for hybridization resulting in available fetal DNA;
(vi) evaluating the cells by fluorescence in situ hybridization (FISH) by contacting the available fetal nucleated DNA with a fluorescent DNA probe hybridizable to a chromosomal fetal DNA associated with a chromosomal abnormality of interest, under hybridization conditions; and
(vii) detecting the presence or absence of hybridization between the fluorescent DNA probe and the chromosomal fetal DNA of interest as an indication of the presence or absence of said chromosomal abnormality.

18. The method according to claim 17 wherein said transcervical cell sample is obtained by cytobrush.

19. The method according to claim 17 wherein said transcervical cell sample is obtained from a pregnant woman at the 6^{th} to 14^{th} week of gestation.

20. The method according to claim 17 wherein the antibody in step (iv) is an antibody specific to the HLA-G antigen expressed only by extravillous trophoblast cells.

21. The method according to claim 17 wherein the chromosomal abnormality is a chromosomal aneuploidy.

22. The method of claim 21 wherein the chromosomal aneuploidy is trisomy 21.

23. The method of claim 21 wherein the chromosomal aneuploidy is trisomy 18.

24. The method of claim 21 wherein the chromosomal aneuploidy is trisomy 13.

25. The method of claim 21 wherein the chromosomal aneuploidy is triploidy
